(19) United States
(12) Patent Application Publication
(10) Pub. No.: US 2024/0156440 A1
(43) Pub. Date: May 16, 2024

(54) METHOD OF RECONSTRUCTING TRANSCRANIAL IMAGES USING DUAL-MODE ULTRASONICS PHASED ARRAY

(71) Applicant: NAVIFUS US LLC, TUSTIN, CA (US)

(72) Inventors: HAO-LI LIU, TAIPEI CITY (TW); HSIANG-CHING LIN, TAIPEI CITY (TW); ZHEN-YUAN LIAO, TAIPEI CITY (TW); HSIANG-YANG MA, TAIPEI CITY (TW); CHIH-HUNG TSAI, KAOHSIUNG CITY (TW); CHUN-HAO CHEN, NEW TAIPEI CITY (TW)

(21) Appl. No.: 18/504,404

(22) Filed: Nov. 8, 2023

Related U.S. Application Data
(60) Provisional application No. 63/383,600, filed on Nov. 14, 2022.

(51) Int. Cl. A61B 8/08; A61B 8/00

(52) U.S. Cl. CPC A61B 8/5207; A61B 8/0808; A61B 8/4245; A61B 8/4488; A61B 8/58

(57) ABSTRACT

A method of reconstructing transcranial images using a dual-mode ultrasonic phased array includes steps of: controlling channels to emit energy toward an intracranial target point of a patient; respectively generating backscattered radiofrequency (RF) data by using the channels to receive backscattered energy reflected from the intracranial target; and reconstructing an acoustic distribution image based on those backscattered RF data in real-time. Compared with Pre-Treatment Ray Tracing Method, the present invention can display intracranial pressure distribution in real-time; compared with MR Thermometry, the present invention can be applied to low-energy applications without temperature change; and compared with Passive Cavitation Imaging, the present invention can stably present acoustic distribution images without relying on microbubbles.

This application claims priority for the U.S. provisional patent application No. 63/383,600 filed on 14 Nov. 2022, the content of which is incorporated by reference in its entirely.

### BACKGROUND OF THE INVENTION

### Field of the Invention

The present invention relates to a method of reconstructing transcranial images using a dual-mode ultrasonic phased array, particularly to a method of real-time presenting transcranial acoustic distribution images.

### Description of the Prior Art

To make Central Nervous System (CNS) therapeutic agents pass through Blood-Brain-Barrier (BBB), reach the desire treatment region, and achieve the expected therapeutic effect, the BBB in the desire region should be disrupted temporarily. Recently, Focused Ultrasonic (FUS) technology has been applied to disrupt the BBB by ultrasonic energy in a non-invasive, localized/selected, and temporary manner. In comparison with modifying a medicine to have liposolubility or injecting chemical substances to enhance the permeability of brain vessels, the FUS technology can locally disrupt BBB of the targeting region and thus prevent medicine from entering the non-targeting region. However, the focused ultrasonic energy must be regulated and monitored repeatedly. Another application of the FUS technology is to regulate the ion channel of the nerve cells, wherein mechanical force (i.e., ultrasonic wave) induces the change of cell membrane potential and thus varies the threshold of nerve operation potential, whereby the diseases (such as the abnormal discharge of epilepsy) may be cured or alleviated. Ultrasonic energy must cooperate with microbubbles via intravenous injection for disrupting the BBB, while no microbubbles need to be injected for application of neuromodulation.

At present, there are several technologies applicable for evaluating or monitoring the intracranial condition in the FUS therapy, such as Pre-Treatment Ray Tracing Method, magnetic resonance thermometry imaging (MRTI) for the Hyperthermia, and Passive Cavitation Imaging (PCI) technology for monitoring BBB opening operation. However, all those technologies respectively have their limitations.

First, Pre-Treatment Ray Tracing Method will be explained. For the purpose of observing the intracranial condition of a patient, the patient must make multiple movements between the magnetic resonance (MR) treatment room and the FUS treatment room, where the MR treatment room should be equipped with the focused ultrasound transducer as well. In addition, the current MR equipment is expensive and bulky, and a single MR scan takes at least 5 minutes. These disadvantages cause inconvenience for the patients, and doctors are unable to observe the intracranial condition in real-time, thus preventing the doctors from instantly taking corresponding operations to adjust the focused point and output energy of the focusing-type ultrasonic transducer.

The MR thermometry imaging features monitoring the temperature variation generated by focused ultrasonic energy using the MR imaging technology. Although the MR thermometry can monitor the result of the transcranial focused ultrasonic energy in real-time, it can only apply to applications with significant temperature variations, such as ablation. For low-energy applications with no temperature changes, such as neuromodulation, it cannot be applied to. Therefore, the MR Thermometry is neither suitable to observe tiny cavitation variations directly nor suitable to predict the result of low-intensity transcranial ultrasonic energy in situ.

The PCI technology features represent the cavitation condition through cavitation characteristics caused by the microbubbles responding to focused ultrasonic energy using the ultrasonic transducer in a passive reception mode where it does not emit but only receives energy. Specifically, the microbubbles are micron-scale particles composed of lipids, albumin, polymers, etc. Due to the high acoustic impedance mismatch and compressibility of microbubbles, microbubbles are compressed by the positive pressure of ultrasonic waves and expanded by the negative pressure of ultrasonic energy in ultrasonic field; this phenomenon is known as the cavitation effect. The cavitation effect is divided into stable cavitation and inertial cavitation. Stable cavitation is that the microbubbles, which are in the ultrasonic field with low-intensity acoustic pressure, will contract and expand with positive and negative pressure periodically and stably. Stable cavitation induce the power levels in the subharmonic wave frequency band (half the ultrasound transducer's driving frequency) and the power levels in the ultra-harmonic wave frequency band (odd harmonic series of the subharmonic) higher than in the non-harmonic frequency band. Inertial cavitation is that the microbubbles in an ultrasonic field at high-intensity acoustic pressure are not synchronous with the positive and negative pressure transitions of the ultrasonic waves, and eventually rupture, resulting in high temperature, high pressure, microinjection pumps, shock waves, broadband radiation, etc. The cavitation characteristics related to the cavitation effect may be applied to the PCI technology, in which the intensity of an image not only can present the position of cavitation but also can predict the condition of cavitation, whereby indirectly presenting the transcranial energy distribution. However, there are disadvantages to the accuracy of imaging based on cavitation features as the followings: leakage of the baseband spectrum and different concentrations of microbubbles affect the potential level of the harmonic waves; the cavitation imaging intensity depends on the cavitation conditions and the concentration of the microbubbles in blood, while the density of microbubbles will gradually decrease and finally disappear, resulting in an unstable cavitation image; the time interval that BBB is deactivated is only few minutes, and the concentration of microbubbles will gradually decrease and finally disappear. In conclusion, although the PCI technology can indirectly present the transcranial energy distribution, the imaging technology limited by the microbubbles fails to provide reliability and stability.

During the low-intensity ultrasonic transcranial therapy, since the emitted energy of the transducer is much smaller than the energy in the hyperthermia, the temperature is almost not increased in the ultrasonic focused point, making temperature monitoring by the MR thermometry impossible. Therefore, while ultrasonic energy is used to disrupt the BBB, microbubbles via intravenous injection are required to induce the cavitation effect, and the ultrasonic feedback signal generated by the cavitation effect are received by the ultrasonic transducer to perform the acoustic source backtrack and cavitation mapping, thereby monitoring the process of BBB opening. However, in neuromodulation or applications without using microbubbles, there is neither a high enough heat source from the temperature rise nor a strong enough sound source from the cavitation effect induced by the microbubbles can be used for monitoring the transcranial focused ultrasonic therapy in the current method.

### SUMMARY OF THE INVENTION

Accordingly, the present invention provides a method of reconstructing transcranial images using a dual-mode ultrasonic phased array, which allows for observation of ultrasonic energy distribution covering a variety of applications such as ultrasonic hyperthermia, the BBB opening, and neuromodulation. The method comprises steps: (A) controlling a plurality of channels to emit energy to an intracranial target point of a patient; (B) respectively generating backscattered radiofrequency (RF) data by using the plurality of channels to receive backscattered energy reflected from the intracranial target point; and (C) reconstructing an acoustic distribution image in real-time based on the backscattered RF data.

In some embodiments, the step of using the backscattered RF data to reconstruct the acoustic distribution image further includes steps: calculating a reconstructed signal according to the backscattered RF data; and reconstructing the acoustic distribution image, which presents energy distribution, according to the reconstructed signal.

In some embodiments, the method further comprises steps: adjusting focuses of the dual-mode ultrasonic phased array, and repeating Step (A) to Step (C) to reconstruct the plurality of acoustic distribution images respectively corresponding to different intracranial target points; and merging the plurality of acoustic distribution images to obtain a tissue image.

In some embodiments, the method further comprises steps: performing Fourier Transform of the backscattered RF data to obtain frequency spectrums; filtering signals of the frequency spectrums to obtain ultra-harmonic signals corresponding to the frequencies output by the dual-mode ultrasonic phased array; and reconstructing a cavitation distribution image according to the ultra-harmonic signals.

In some embodiments, the method further comprises a step: integrating the energy, which the dual-mode ultrasonic phased array outputs to the intracranial target point, an elapsed time after applying energy, and the acoustic distribution image to work out an intracranial temperature increase and an intracranial temperature decrease, which are induced by acoustic energy, for reconstructing an intracranial temperature distribution image.

The method of constructing transcranial images of the present invention uses a dual-mode ultrasonic phased array to transcranially monitor the energy of the focused ultrasonic technology, outperforming the pre-treatment ray-tracing method in equipment cost, occupied area, and imaging speed. The present invention passively receives backscattered ultrasonic signals, exempted from using microbubbles. The present invention undertakes reconstruction of passive images and then evaluates transcranial pressure distribution. After the phase calibration of reconstruction, the transcranial pressure distribution highly correlates with the intensity of the baseband passive image. In the case where no microbubble is injected, the present invention can still be correctly positioned at the region of interest (ROI). Further, the energy distribution image of the backscattered signals can present the focused position and the magnitude of ultrasonic energy in real-time after phase calibration or energy adjustment.

In conclusion, the present invention has the following advantages: compared with MRI, the present invention can display intracranial pressure distribution in real-time through the skull; compared with MR Thermometry, the present invention can be applied to low-energy applications without temperature change; and compared with PCI, the present invention can stably present energy distribution images without relying on microbubbles.

### BRIEF DESCRIPTION OF THE DRAWINGS

FIG. 1 is a diagram schematically showing a dual-mode ultrasonic phased array according the present invention.

FIG. 2 is a diagram schematically showing that a plurality of channels emits energy to an intracranial target point.

FIG. 3 shows a flowchart of a method of constructing transcranial images according the present invention.

FIG. 4 shows an acoustic distribution image reconstructed by the present invention.

FIG. 5 shows an image obtained with a hydrophone.

FIG. 6 shows a flowchart of a method of constructing transcranial images according to some embodiments of the present invention.

FIG. 7 schematically shows that a cranial bone deviates the energy emitted by the channels according to one embodiment of the present invention.

FIG. 8 and FIG. 9 show the images before and after focus calibration, respectively.

FIG. 10 is a diagram showing a single-side frequency spectrum of backscattered RF data.

### DETAILED DESCRIPTION OF THE INVENTION

The embodiments of the present invention will be further demonstrated in details hereinafter in cooperation with the corresponding drawings. In the drawings and the specification, the same numerals represent the same or the like elements as much as possible. For simplicity and convenient labelling, the shapes and thicknesses of the elements may be exaggerated in the drawings. It is easily understood: the elements belonging to the conventional technologies and well known by the persons skilled in the art may be not particularly depicted in the drawings or described in the specification. Various modifications and variations made by the persons skilled in the art according to the contents of the present invention are to be included by the scope of the present invention.

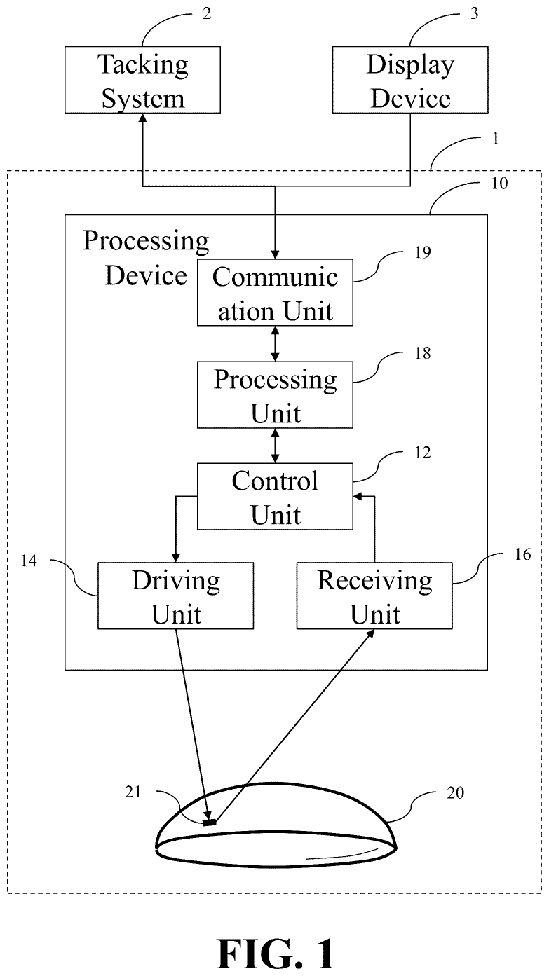

The present invention provides a method of reconstructing transcranial images using a dual-mode ultrasonic phased array (for instance, transcranial dual-mode ultrasonic phased array), which is used to reconstruct transcranial acoustic distribution images in real-time. The acoustic distribution images may be but are not limited to be energy distribution images, tissue images, cavitation images, and temperature distribution images. Refer to FIG. 1. The dual-mode ultrasonic phased array 1 used by the present invention comprises a processing device 10 and a plurality of transduction elements (i.e., a plurality of channels 21 of the ultrasonic transducer) forming an array on a casing 20. For simplicity, only a channel 21 is used to exemplify the channels in FIG. 1. The processing device 10 includes a control unit 12, a driving unit 14, a receiving unit 16, and a processing unit 18. The processing unit 18 is in signal communication with the control unit 12 to controls the transmission and reception of the channels 21 and is in signal communication with the receiving unit 16 to acquire the data generated by controlling the channels 21. “Dual-mode” indicates that the channels 21 may function to transmit and receive. The control unit 12 is in signal communication with the channels 21, driving the transmission and reception of the channels 21 according to the instructions of the processing unit 18. The channels 21 respond to the electric field applied by the driving unit 14 to generate acoustic waves and respond to acoustic waves to generate signals representing the magnitudes of energy to the receiving unit 16. In some embodiments, to enable doctors to view the images in real-time, the processing device 10 further includes a communication unit 19, which is in signal communication with the processing unit 18. The communication unit 19 is directly/indirectly connected with a display device 3 in wired/wireless signal communication to present the images.

To present the relative position of the dual-mode ultrasonic phased array and the head of the patient in real-time, the dual-mode ultrasonic phased array of the present invention may further incorporate a tracking system 2, which is used to track and guide, whereby to realize the method of the present invention. The tacking system 2 may be realized by at least one of an optical method, an electromagnetic method, and an acoustic method. Specifically, the tracking system is an optical positioning-tracking system, which includes an infrared transceiver for emitting or receiving light and a charge-coupled device (CCD) image capturing device, or a depth camera (also called the camera). The implementation steps of the optical positioning tracking system are sequentially an establishing step, a registering step, and a tracking step. The establishing step is to construct or prepare 3D computer models of the target objects. The target objects are the head of the patient and the dual-mode ultrasonic phased array. The 3D model of the patient's head may be realized via a radiological 3D imaging technology, such as the Magnetic Resonance Imaging (MRI) technology, the Computed Tomography (CT) technology, the Positron Emission Tomography (PET) technology, or the Single Photon Emission CT (SPECT) technology. The registering step is to acquire the relative position, orientation, and focused position of the dual-mode ultrasonic phased array and obtain the polygon mesh of the patient's head. Specifically, a physical model of the dual-mode ultrasonic phased array having a needle and a plurality of patterned labels attached onto the casing is provided. The tip of the needle is to stimulate the focused point. Next, an image capturing device is used to capture the image of the abovementioned model, and the processing unit 18 recognizes the patterned labels and the tip using an image recognition technology, whereby to obtain the space information of the dual-mode ultrasonic phased array 1, such as the relative position, the orientation, and the focused position. Then, while the tip of the model slides over the patient's head that is also stuck with patterned labels, the space information is persistently collected to construct the data of the polygon mesh of the patient's head. Finally, the 3D model of the patient's head is integrated with the abovementioned data. Depending on requirements, the tracking step is to apply the space information and the data obtained in the registering step to the dual-mode ultrasonic phased array and the method using the same. The optical positioning-tracking system, which is used to replace the MRI to output corresponding positional information, may obtain the coordinates of the cranium and the coordinates of all the channels within few seconds, whereby the data of phase calibration may be worked out. The optical positioning-tracking system and the dual-mode ultrasonic phased array occupy less space than the MRI equipment and are free from the limitations that constrain the MRI technology. Alternatively, the abovementioned tracking system may adopt an electromagnetic navigation technology.

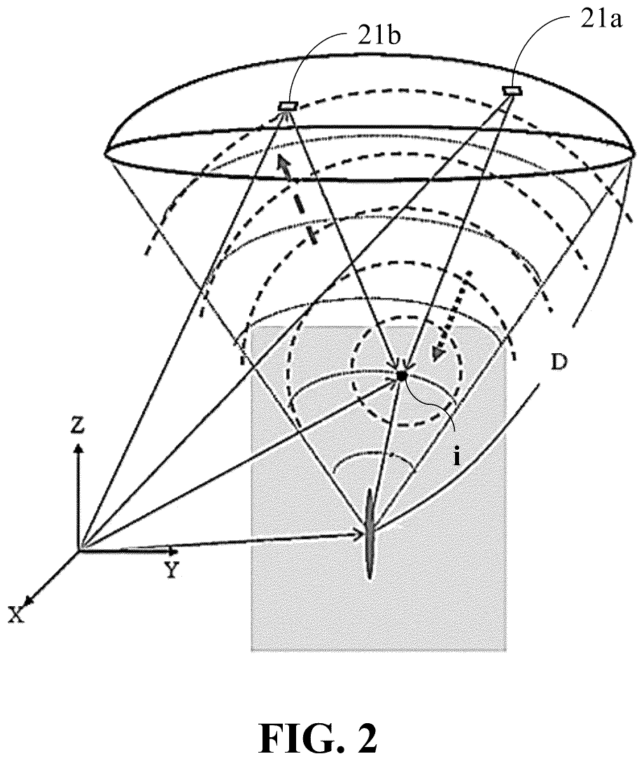

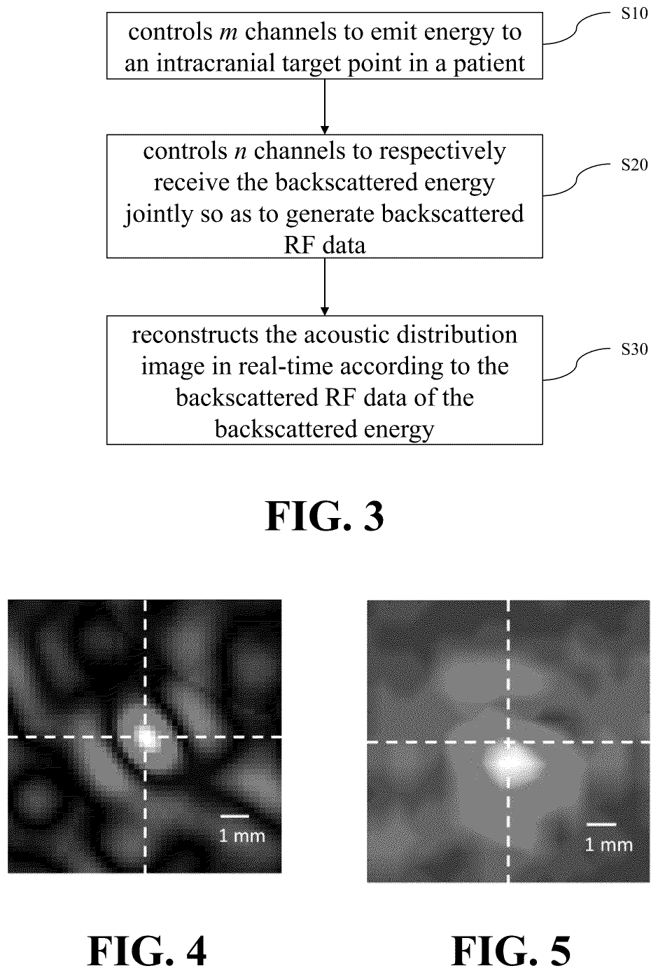

First, the process of using the dual-mode ultrasonic phased array to reconstruct the energy distribution image in real-time will be explained. Refer to FIG. 2 and FIG. 3 simultaneously. FIG. 2 schematically shows that several channels transmit energy to and receive energy from the target point inside the cranium. In FIG. 2, the cranium is omitted, and a gray block is used to represent the brain. FIG. 3 shows the flowchart of the method of constructing transcranial images according to one embodiment of the present invention. In Step S10, the processing device controls m channels 21a (only a representative channel 21a is depicted in FIG. 2) to emit energy to an intracranial target point, wherein m is a positive integer larger than or equal to 2. The intracranial target point i may be a nidus area inside the patient's cranium, such as the local area where BBB has been disrupted by the focused ultrasonic energy. In practical application, the intracranial target point i may be a non-nidus area. The energy emitted by each of the channels 21a (denoted by dotted lines) will decay and deviate during the process that the energy passes through the cranium and reaches the intracranial target point i. Then, the energy is partially reflected by the intracranial target point i to form longitudinal waves toward multiple directions. The longitudinal waves interfere with each other to generate backscattered energy (denoted by dashed arrows). In Step S20, the processing device controls n channels 21b (only a representative channel 21b is depicted in FIG. 2) to respectively receive the backscattered energy jointly generated by the in channels 21a so as to generate backscattered RF data, wherein n is a positive integer larger than or equal to 2. Then, in Step S30, the processing device reconstructs the acoustic distribution image in real-time according to the backscattered RF data of the backscattered energy. As shown in FIG. 4, the acoustic distribution image can present the degree of the acoustic pressure distribution at the intracranial target point i corresponding to transcranial energy, commonly referred to as Transcranial Pressure Distribution.

Since the abovementioned n channels 21b respectively receive different amounts of the backscattered energy, the corresponding generated backscattered RF data are different. The plurality of backscattered RF data is used to reconstruct the acoustic pressure distribution. By selecting the appropriate colors or brightness according to the size of the backscattered RF data, the acoustic distribution imaging can visually present the location and intensity of the acoustic distribution in an intuitive manner. Besides, digital image technologies such as the interpolation method can further optimize the quality and resolution of the acoustic distribution image.

To evaluate the quality of the acoustic distribution image of the present invention, a polyvinylidene difluoride (PVDF) hydrophone is used as the standard. Refer to FIG. 4 and FIG. 5 simultaneously. FIG. 4 is the acoustic distribution image reconstructed by the present invention. FIG. 5 is the image of the transcranial pressure distribution obtained by using the PVDF hydrophone to measure the intracranial target point. The image in FIG. 5 has been phase-calibrated. In FIG. 4 and FIG. 5, the level of brightness is proportional to the magnitude of the acoustic pressure. The resolution of the image generated by the present invention is clear and similar to that of the energy distribution image obtained by the PVDF hydrophone.

As mentioned above, the PCI technology may inspect the intracranial cavitation characteristics, such as position and condition, via injecting microbubbles. Although the cavitation characteristics may indirectly represent the transcranial pressure distribution, the following disadvantages exist: (1) the spectral leakage of the baseband and different densities of microbubbles may affect the level of the harmonic signal; (2) the concentration of the microbubbles in the blood may gradually decrease and finally disappear because of the metabolism and the ultrasonic applied to the microbubbles, resulting in instability of the cavitation image generated by the cavitation characteristics; and (3) after BBB is successfully disrupted, only few minutes are available for inspection. In comparison with the PCI technology, the present invention doesn't rely on microbubbles but can perform the calculation of reconstruction and evaluate the transcranial pressure distribution based on the backscattered energy. Therefore, the present invention can be exempted from the abovementioned disadvantages.

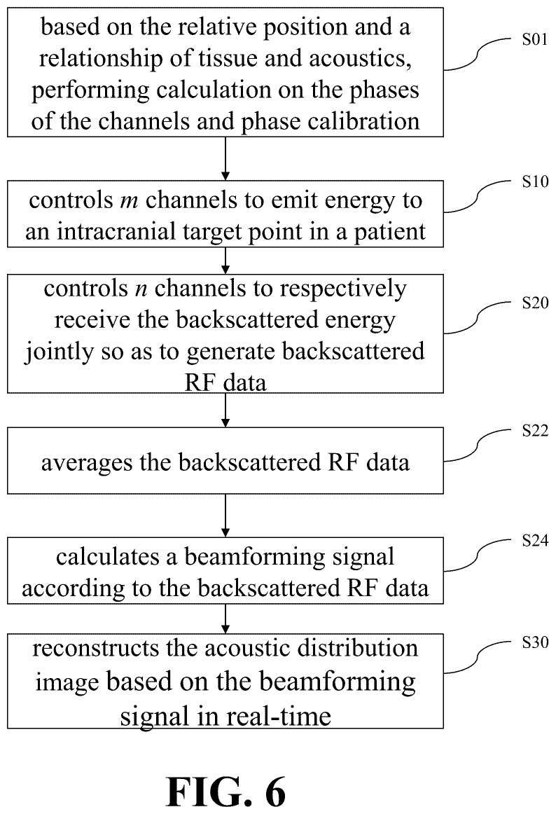

In some embodiments, the acoustic distribution image is a kind of energy distribution image. Refer to FIG. 6. In detail, during the process of reconstructing the acoustic distribution image, the processing device calculates a reconstructed signal according to the backscattered RF data beforehand (Step S24). Next, based on the reconstructed signal, the processing device reconstructs the acoustic distribution image, which presents the specific location and energy distribution of the dual-mode ultrasound array transducer focused on the intracranial target point (Step S30). Preferably, the processing device obtains the reconstructed signal according to Equation (1):

S(rᵢ)=Σₙ₌₁^{N}Aₙᵢ×sₙ(rₙᵢ, tᵢ) (1)

wherein S(rᵢ) represents the value calculated by the processing device based on the backscattered RF data generated by the n channels; rᵢ represents the positions of the intracranial target points; Aₙᵢ respectively represent the weights of the n channels; sₙ respectively represent the backscattered RF data individually generated by the n channels after receiving the backscattered energy; rₙᵢ respectively represent the distance between each of the n channels and the intracranial target point i; tᵢ represents the time delay by which each of the n channels receives energy. According to Equation (1) of the reconstruction technology, the processing device calculates the distance rₙᵢ between each of the n channels and the intracranial target point i, the time delays tᵢ, and the weights Aₙᵢ to obtain the results, which correspond to the plurality of the energy respectively output by the in channels at the same time point, for reconstructing the transcranial pressure distribution. However, the present invention is not limited by the above description. For practical requirements, in some embodiments, the in channels may emit energies at different time points, and the processing device can still obtain the same result, although the time points are different. In other words, before reconstructing the energy distribution image, the present invention respectively calculates the time delays of the in channels and/or the n channels to obtain the expected calculation results.

The ultrasonic energy may undergo distortion, twisting, or decay while passing through the cranial bone, reducing the quality and reliability of the acoustic distribution image reconstructed based on the backscattered RF data. Refer to FIG. 6. In some embodiments, before reconstructing the acoustic distribution image, the processing device averages the backscattered RF data individually generated by the n channels that receive the backscattered energy (Step S22) so as to enhance the quality of the acoustic distribution image.

In detail, the processing device performs Discrete Fourier Transform (DFT) on the input signals according to Equation (2) at first, and it also be applied to compensate for the variable of the time delay so as to acquire the frequency-domain signal Sₐ(k).

$$\\ \begin{matrix}{S}_{a} ( k ) = {\sum }_{n = 0}^{N - 1} {s}_{a} ( n ) {e}^{{\frac{2 π {i}^{}}{n}}^{( n - 1 ) ( k - 1 )}} & ( 2 )\end{matrix}$$

wherein sₐ(n) represents the input signal, i.e., the backscattered RF data generated by one of the n channels; k represents each discrete frequency point in the frequency domain, normally ranging from 0 to N−1; N represents the number of the discrete sampled points in the input signal.

Next, the processing device repeats the abovementioned step until the processing device obtains all the frequency-domain signals corresponding to the n channels, which are respectively expressed by Sₐ(k), S_{b}(k) . . . Sₙ(k).

Next, the processing device performs cross-correlation on the frequency-domain signal (such as Sₐ(k)) of one of the n channels and the frequency-domain signal (such as S_{b}(k)) of another one of the n channels to obtain S_{corr[a,b]} according to Equation (3):

S_{corr[a,b]}=Sₐ(k)·S_{b}(k) (3)

Next, the processing device repeats the abovementioned step until the processing device acquires the cross-correlations of Sₐ(k) and each of the frequency-domain signals of the n channels, i.e., S_{corr[a,b]}, S_{corr[a,c]} . . . S_{corr[a,n]}. Then, the processing device averages the post-cross-correlation signals to obtain S_{corr}(k). Thus, the phased aberration is reduced while the signals corresponding to the n channels pass through the cranial bone, completing the spectral aberration averaging.

Next, the processing device performs the cross-correlation and averaging on the frequency-domain signals S_{b}(k) . . . Sₙ(k) corresponding to the rest of the n channels, thereby completing the spectral aberration averaging for all the channels.

Next, according to Equation (4), the processing device performs Inverse Discrete Fourier Transform (IDFT) on S_{corr}(k) to obtain the final signals of the time domain.

$$\\ \begin{matrix}{s}_{corr} ( n ) = {\sum }_{k = 1}^{n} {S}_{corr} ( k ) {e}^{{\frac{2 π {i}^{}}{n}}^{- ( n - 1 ) ( k - 1 )}} & ( 4 )\end{matrix}$$

Next, according to Equation (5), the processing device finds out the maximum value from those s_{corr}, and performs calculation on the maximum s_{corr} and another s_{corr} to obtain the time delay t_{delay[a,b]} of a given channel with respect to the maximum s_{corr}. The acquired time delay may be used to eliminate the phase difference between the channels.

t_{delay[a,b]}=max(x_{corr[a,b]})−N/2 (5)

wherein N is the total quantity of the channels receiving the backscattered energy.

Next, the processing device repeats the abovementioned steps until the processing device acquires the time delays of all the channels with respect to the maximum s_{corr}.

Those s_{corr}(n) and/or those t_{delay[a,b]} may be adapted to Equation (1) for calculating reconstructed signals so as to compensate for the decay of the backscattered energy and/or the phase difference of the backscattered RF data, which results from the relative time delay of the channels, whereby to enhance the quality of the energy distribution image. For example, while t_{delay[a,b]} of a channel is greater than 0, the phase of the backscattered RF data generated by the channel is shifted forward; while t_{delay[a,b]} of a channel is smaller than 0, the abovementioned phase is shifted backward.

As mentioned above, ultrasonic energy will be defocused or deviated while passing the cranial bone, which not only affects the quality of the acoustic distribution image but also defocuses or deviates the focused position of ultrasonic energy. Therefore, the present invention also provides a phase calibration method: performing phase calibration of the m channels emitting energy based on the difference between the real phase and the expected phase of the dual-mode ultrasonic phased array. Refer to FIG. 6. As shown in Step S01, before reconstructing the acoustic distribution image, the method further comprises the following steps: acquiring the relative position between the dual-mode ultrasonic phased array and the patient's head; based on the relative position and a relationship of tissue and acoustics, performing calculation on the phases of the channels. The relative position may be acquired using the abovementioned optical positioning-tracking system. The relationship of tissue and acoustics is obtained via the difference between the transmitted information and the received information of the dual-mode ultrasonic phased array, including the scatted vectors entering or leaving the cranial bone, the density of the cranial bone, and the speed of the acoustic wave inside the cranial bone. It is known that two refractions of acoustic waves occur during the process where the acoustic wave is transmitted from the dual-mode ultrasonic phased array to the intracranial target point. The first refraction occurs (at the external side of the cranial bone) when the ultrasonic wave enters the cranial bone. The second refraction occurs (at the internal side of the cranial bone) when the ultrasonic wave leaves the cranial bone. Regarding the defocusing and deviation of the focused position due to the two refractions, the processing device may acquire the physical phases of the m channels according to the methods involving the scattered vectors, the cranial bone density, and the intracranial acoustic velocity described thereinafter. Thereby, the processing device may further calculate the difference between the physical phase and the expected phase and then complete the phase calibration for the m channels in real-time.

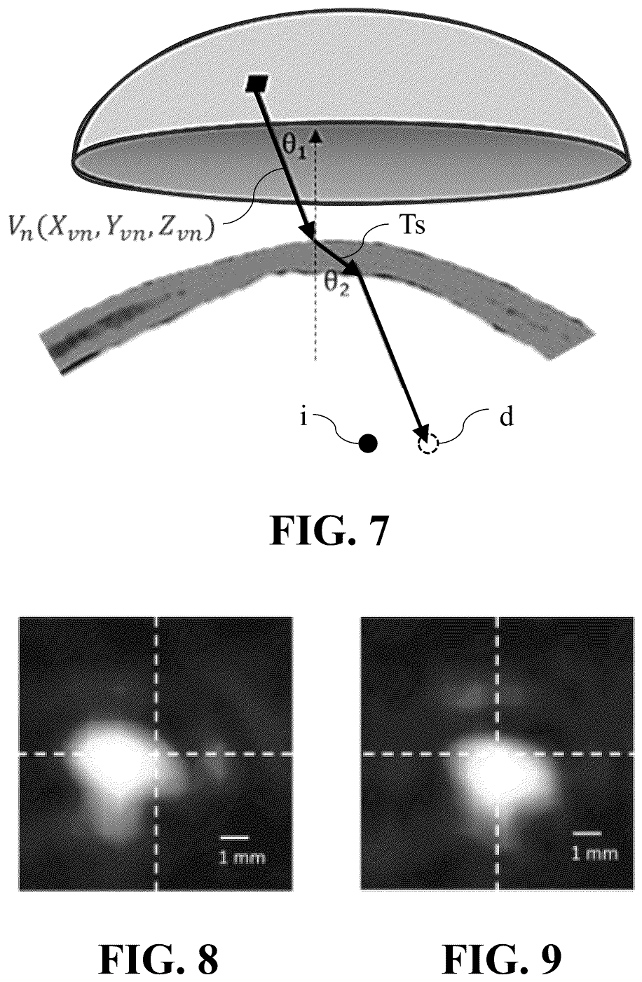

Refer to FIG. 7, which illustrates how the energy emitted by the channels deviates from the intracranial target point i to the displacement point d due to the cranial bone. In the first refraction, the scattered vector Tₛ represents the intracranial path of the ultrasonic energy having entered the cranial bone. The scattered vector Tₛ may be obtained using Equation (6) and Equation (7) of the Snell's law:

$$\\ \begin{matrix}e = \frac{{C}_{i}}{{C}_{r}} & ( 6 )\end{matrix}$$

$$\\ \begin{matrix}{T}_{s} = \frac{V}{e} + N * ( \frac{Cos ( {θ}_{1} )}{e} - Cos ( {θ}_{2} ) ) & ( 7 )\end{matrix}$$

wherein e represents the acoustic velocity ratio; Cᵢ represents the velocity of the acoustic wave in the incident medium; Cᵣ represents the velocity of the acoustic wave in the refracting medium (the cranial bone); Vₙ represents the vector of acoustic energy from the emitting channels to the outer side of the cranial bone and is simplified as V, wherein N represents the normal vector; θ₁ represents the incident angle; θ₂ represents the refraction angle.

In one embodiment, the cranial bone density ρ may be learned from the statistic record. However, the present invention is not limited by the embodiment. It is preferred that before performing calculation of the phase of the channels, the values of the Hounsfield Unit (HU), which are measured in the CT of the patient, may be used to calculate the cranial bone density p of the patient according to Equation (8):

$$\\ \begin{matrix}ρ = {ρ}_{min} + \frac{( {ρ}_{max} - {ρ}_{min} ) * ( HU - {HU}_{min} )}{( {HU}_{max} - {HU}_{min} )} & ( 8 )\end{matrix}$$

wherein ρₘᵢₙ represents the density of water; ρₘₐₓ represents the maximum density of a healthy person, such as 2700 Kg/m 3; HU, HUₘₐₓ, and HUₘᵢₙ respectively represent the standard value, the maximum value, and the minimum value of the Hounsfield Unit, which are measured in CT, wherein HU is a variable varying according to the measured position; HUₘₐₓ and HUₘᵢₙ are the fixed values in the Hounsfield Scale.

In one embodiment, the intracranial acoustic velocity c may be learned from the statistic record. However, the present invention is not limited by the embodiment. The abovementioned cranial bone density ρ may be introduced into Equation (9) to calculate the intracranial acoustic velocity c.

$$\\ \begin{matrix}c = {c}_{min} + \frac{( {c}_{max} - {c}_{min} ) * ( ρ - {ρ}_{min} )}{( {ρ}_{max} - {ρ}_{min} )} & ( 9 )\end{matrix}$$

wherein c miii represents the acoustic velocity in water; cₘₐₓ represents the acoustic velocity corresponding to ρₘₐₓ, such as 4000 m/s.

Based on the above discussion and through the calculation of the first and second refractions, the processing device can obtain the energy scattering directions at the intracranial target position, wherein the energies are emitted by the channels and pass through the cranium bone. Further, the processing device may work out the time of flight based on the acoustic velocity. According to the scattering directions and the time of flight, the processing device may perform phase calibration for all the channels. Refer to FIG. 8 and FIG. 9, which respectively show the focused points before and after the real-time focus calibration. Those shown in FIG. 8 and FIG. 9 are not the acoustic distribution images of the present invention but the images of the PVDF hydrophone and are only to present the difference between the images captured before calibration and after calibration.

In other words, the fundamental-frequency passive energy distribution image, obtained after the phase calibration based on the relative position acquired by the tracking system (such as the navigator using radiation in tracking), may be used to evaluate the transcranial acoustic pressure distribution. It is because the fundamental-frequency passive energy distribution image highly correlates with the transcranial acoustic pressure distribution.

In some embodiments, after the step of reconstructing the acoustic distribution image, the method further comprises the following step: adjusting the energy output by the dual-mode ultrasonic phased array based on the acoustic distribution image. In detail, the processing device may calculate the transcranial decay rate and the compensation parameter based on the acoustic distribution image, which represents the reflected energy, so as to obtain the energy physically transmitted to the intracranial target point. The difference between the physical energy value and the expected energy value of the dual-mode ultrasonic phased array may function as the reference for adjusting the energy.

In some embodiments, the channels include a plurality of first channels and a plurality of second channels. The first channels are used to perform the step of emitting energy to the intracranial target point. The second channels are used to perform the step of receiving the backscattered energy. For example, there are 256 channels, of which 128 channels are used to emit energy to the intracranial target point, and the residual 128 channels are used to persistently receive the scattered energy generated by the abovementioned energy. The quantity of the channels responsible for emitting energy and the quantity of the channels responsible for receiving energy may be adjusted according to practical requirements. However, the present invention is not limited by the embodiments. Responding to the processing device, these channels may exchange to perform the step of emitting energy to the intracranial target point and the step of receiving backscattered energy alternately by an interval of milliseconds. In other words, the channels for emitting energy to the intracranial target point and the channels for receiving backscattered energy may be the same channels.

In some embodiments, the acoustic distribution image is a tissue image. In detail, the processing device persistently adjusts the phases of the channels for real-time focus calibration, thereby reconstructing the acoustic distribution images of multiple intracranial regions while the dual-mode ultrasonic phased array is scanning the intracranial space. Then, the processing device merge the acoustic distribution images to obtain tissue images using the panoramic imaging technology or the image overlay technology. It is because different tissues may scatter out different intensities of energy for a given magnitude of energy according to their acoustic characteristics. Whether the tissues are the same tissues may be determined according to whether the levels of brightness of the scanned areas are the same. The scanning is initiated at the geometric focus and then proceeds by moving around and outward from the geometric focus. For example, the scanning is offset by 3 mm and a pulse emitted per 120 degrees in rotations; the scanning is offset by 6 mm and a pulse emitted per 30 degrees in rotations; the scanning is offset by 9 mm and a pulse emitted per 15 degrees in rotations. It is preferred that the acoustic distribution images may be obtained via the abovementioned method of generating energy distribution images. In other words, a single energy distribution image can present the transcranial pressure distribution of the intracranial target point; a plurality of energy distribution images of multiple areas may be merged to form the tissue image, which can fully present the intracranial structure. It is preferred that the tissue images may be integrated with the 3D model of the patient's head so as to present the tissue images on the display device in a three-dimensional manner.

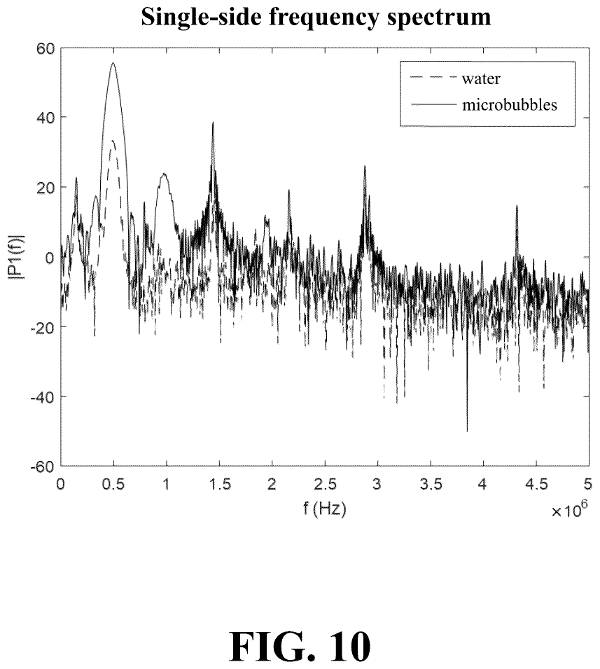

Different from the PCI technology, although the transcranial imaging method of the present invention can reconstruct the intracranial acoustic pressure distribution without using the cavitation effect of microbubbles, the method can still be used to observe the condition of cavitation. In some embodiments, the acoustic distribution image is a cavitation distribution image. In detail, when microbubbles exist in the intracranial target point, the energy emitted by the m channels will make the microbubbles generate the cavitation effect, resulting in the frequency spectrum obtained by the processing device after performing Fourier Transform on the backscattered RF data generated by the n channels will reflect some physical characteristics. Refer to FIG. 10, which shows the positive frequency portion of the backscattered RF data X(t), i.e., the single-side frequency spectrum. Suppose that the frequency of the output ultrasonic energy is 0.5 MHz and that water is used to simulate the brain tissue. When microbubbles do not exist, only 0.5 MHz signal and noise (as shown in the dashed lines) appear in the frequency spectrum; conversely, when microbubbles exist, because the microbubbles will generate 0.5 MHz baseband and the ultra-harmonic signal having the integral times of frequencies in the acoustic field of 0.5 MHz, the spectrum diagram not only contains the aforementioned noise but also exhibits a prominently elevated signal at 0.5 MHz due to interference effects and signals corresponding to ultra-harmonics (as shown in the solid lines). Given the significant difference between the signal and noise at the 1 MHz, the processing device may filter out 1 MHz and reconstruct the cavitation distribution image thereby. If dark zones appear in the reconstructed image, it indicates that no cavitation effect occurs. If bright zones appear in the reconstructed image, it indicates that the cavitation effect occurs. It is preferred to integrate the cavitation distribution image with the energy distribution image to monitor the transcranial acoustic pressure distribution more safely and effectively.

Different from the MR Thermometry, the present invention is applicable of monitoring not only applications with significant temperature variations but also low-energy applications with no temperature changes. In some embodiments, the acoustic distribution image is an image correlating with temperature distribution. The relationship of the applied ultrasonic energy and the temperature variation of the human tissue or bone is expressed by Equation (10):

$$\\ \begin{matrix}Δ {T}_{L} = \frac{τ 2 α I}{{c}_{v}} & ( 10 )\end{matrix}$$

wherein ΔT_{L} represents the maximum temperature variation caused by the applied ultrasonic energy; τ represents the perfusion time constant, such as 109 seconds for the brain and 3260-6600 seconds for the marrow; α represents the absorption coefficient of the tissue, which is used to evaluate the extent of the absorption of ultrasonic waves or another wave-type energy by a unit length of medium or a unit volume of medium; I represents the energy acting on the tissue, i.e., the intensity of the ultrasonic energy in the abovementioned acoustic distribution image expressing the distribution of energy; Cᵥ represents the heat capacity. After acquiring ΔT_{L}, the processing device can work out the current temperature at the intracranial target point (the biological tissue) based on ΔT_{L} and the elapsing time. During the period of applying the focused ultrasonic energy, the processing device uses the elapsed time t to calculate the temperature increase of the biological tissue according to Equation (11):

ΔT=ΔT_{L}[1−e^{-t/τ}]for 0<t<t′ (11)

wherein t′ represents the time used to reach the highest temperature. After stopping applying ultrasonic energy, the processing device can work out the temperature decrease of the biological tissue according to Equation (12):

ΔT=ΔT_{L}[−e^{-(t-t′)/τ}]for t′<t (12)

According to the temperature increase and the temperature decrease, the processing device can reconstruct the temperature distribution image of the intracranial target point. In other words, the present invention integrates the temperature increase and the temperature decrease with the energy distribution image to reconstruct the temperature distribution image of the intracranial target point, whereby the transcranial acoustic pressure distribution can be monitored more safely and effectively.

The embodiments described above are only to exemplify the present invention but not to limit the scope of the present invention. The embodiments involving equivalent replacement or variation made easily according to the technical contents disclosed by the specification or claims are to be also included by the scope of the present invention.

What is claimed is:

1. A method of reconstructing a transcranial image using a dual-mode ultrasonic phased array, applicable to a processing device in signal communication with a plurality of channels forming an array on the dual-mode ultrasonic phased array, the method comprising steps:
Step (A): controlling the plurality of channels to emit energy to an intracranial target point of a patient;
Step (B): respectively generating backscattered radiofrequency (RF) data by using the plurality of channels to receive backscattered energy reflected from the intracranial target point; and
Step (C): reconstructing an acoustic distribution image in real-time based on the backscattered RF data.

2. The method according to claim 1, wherein before the step of reconstructing the acoustic distribution image, the method further comprises steps:
acquiring a relative position between the dual-mode ultrasonic phased array and the patient's head; and
calculating phases of the plurality of channels emitting energy to the intracranial target point based on the relative position and a relationship of tissue and acoustics.

3. The method according to claim 2, wherein before the step of calculating the phases of the plurality of channels emitting energy to the intracranial target point, the method further comprises a step:
calculating a density of intracranial tissue based on Hounsfield Unit (HU) values of a computer tomography corresponding to the patient to acquire the relationship of tissue and acoustics.

4. The method according to claim 3, wherein after the step of calculating the phases of the plurality of channels emitting energy to the intracranial target point, the method further comprises a step: performing phase calibration on the plurality of channels emitting energy according to the relative position of the dual-mode ultrasonic phased array and the patient's head and the relationship of tissue and acoustics. 5. The method according to claim 1, wherein after the step of reconstructing the acoustic distribution image, the method further comprises a step: adjusting energy output by the dual-mode ultrasonic phased array according to the acoustic distribution image.

6. The method according to claim 5, wherein the processing device calculates a transcranial decay rate and a compensation parameter according to reflected energy so as to adjust the energy output in real-time.

7. The method according to claim 1, wherein before the step of reconstructing the acoustic distribution image, the method further comprises a step: calculating delay times respectively corresponding to the plurality of channels, the delay times being used to compensate the backscattered RF data.

8. The method according to claim 1, wherein before the step of reconstructing the acoustic distribution image, the method further comprises a step: averaging the backscattered RF data to enhance a quality of the acoustic distribution image.

9. The method according to claim 1, further comprising steps:
performing Fourier Transform on the backscattered RF data to obtain frequency spectrums; filtering signals of the frequency spectrums to obtain ultra-harmonic signals of frequencies output by the dual-mode ultrasonic phased array; and
reconstructing a cavitation distribution image according to the ultra-harmonic signals.

10. The method according to claim 1, wherein the plurality of channels includes a plurality of first channels and a plurality of second channels; the plurality of first channels is used to perform the step of emitting energy to the intracranial target point; the plurality of second channels is used to perform the step of receiving the backscattered energy.

11. The method according to claim 1, wherein the plurality of channels is configured to perform the step of emitting energy to the intracranial target point and the step of receiving the backscattered energy respectively at different time points.

12. The method according to claim 1, wherein the step of reconstructing the acoustic distribution image in real-time based on the backscattered RF data further includes steps: calculating a reconstructed signal according to the backscattered RF data; and reconstructing the acoustic distribution image, which presents energy distribution, according to the reconstructed signal.

13. The method according to claim 12, wherein the reconstructed signal is obtained according to Equation (1):
S(rᵢ)=Σₙ₌₁^{N}Aₙᵢ×sₙ(rₙᵢ, tᵢ) (1)
wherein S(rᵢ) represents the reconstructed signal corresponding to the intracranial target points rᵢ; Aₙᵢ respectively represent a weight coefficient of the nth channel; sₙ(rₙᵢ, tᵢ) represents the backscattered RF data generated by the nth channel; rₙᵢ respectively represent the distance between the nth channel and the intracranial target point; tᵢ represents a time interval between a time point at which the nth channel emits energy and a time point at which the nth channel receives the backscattered energy; n is a positive integer.

14. The method according to claim 12, further comprising steps:
adjusting focuses of the dual-mode ultrasonic phased array, and repeating Step (A) to Step (C) to reconstruct a plurality of acoustic distribution images respectively corresponding to different intracranial target points; and
merging the plurality of acoustic distribution images to obtain a tissue image.

15. The method according to claim 12, further comprising steps:
integrating an output energy of the dual-mode ultrasonic phased array, an elapsed time after applying energy, and the acoustic distribution image, which presents energy distribution, to work out a temperature increase and a temperature decrease of the intracranial target point; and
reconstructing a temperature distribution image of the intracranial target point according to the temperature increase and the temperature decrease.
